(19) United States
(12) Patent Application Publication
(10) Pub. No.: US 2005/0251101 A1
(43) Pub. Date: Nov. 10, 2005

(54) Packaged hygiene product for receiving body fluids and/or excretions and process for the production of such a product

(76) Inventor: Albert Becker, Ehlscheid (DE)

Correspondence Address: Rosenthal & Osha L.L.P., Suite 2800, 1221 McKinney Street, Houston, TX, 77010, US

(21) Appl. No.: 10/663,201

(22) Filed: Sep. 16, 2003

(30) Foreign Application Priority Data
Sep. 17, 2002 (DE) 102 43 156.6

(51) Int. Cl. A61F 13/15; A61F 13/20

(52) U.S. Cl. 604/385.01; 604/385.02

(57) ABSTRACT

Described is a packaged hygiene product for receiving body fluids and/or excretions, wherein the hygiene product has a body side facing towards the body and a washing side facing away from the body and can be folded or rolled up for packaging, wherein an adhesive is disposed at least partially at the washing side and wherein there is provided a packaging material which has an inside facing towards the hygiene product and an outside facing away from the hygiene product, wherein the washing side of the hygiene product is fixed to the inside by means of the adhesive. The aim is to develop that hygiene product in such a way that on the one hand the fish mouth effect is avoided and on the other both the packaged hygiene product itself is cheaper to produce and also the machines for the production of the packaged hygiene products are simplified. To attain that object it is proposed that provided in the packaging material is at least one opening through which a surface region of the inside can be fixed to the washing side by means of a part of the adhesive in order to hold the hygiene product in the packaged condition.

### I. FIELD OF USE

The present invention concerns a packaged hygiene product for receiving body fluids and/or excretions and a process for the production of such a product. The term hygiene product is used in accordance with the present invention to denote in particular panty liners, sanitary towels, diapers, paper handkerchiefs and similar products.

### II. TECHNICAL BACKGROUND

It is known for folded-up sanitary towels to be held in the packaged condition merely by means of the side impressions of the packaging material enclosing the sanitary towel. In that case a fish mouth effect frequently occurs, in which the packaging material, along its outwardly disposed end edge which extends between the two side impressions, undesirably lifts away from that region of the packaging material which is below that end edge and against which the end edge initially loosely bears, by virtue of resilient forces of the packaging material. That results in a tunnel-like opening which is laterally delimited by the side impressions and which is similar in its appearance to the mouth of a fish.

A packaged sanitary towel in which the fish mouth effect is substantially avoided is known for example from FIG. 11 of U.S. Pat. No. 6, 234,229. That sanitary towel in the folded-up or folded-together condition is enclosed by a packaging material which is held in the closed condition by an adhesive band strip (tape). As an alternative to the adhesive tape it is also known for the mutually overlapping end regions of the packaging material to be glued together by means of a separate application of glue, thereby to avoid the fish mouth effect.

The known end closures in the form of the adhesive tape or the separate application of glue admittedly avoid the fish mouth effect but they do entail various disadvantages. They require the use of additional material, namely either the adhesive tape or the separate glue. The latter makes itself felt not only in the price of the packaged hygiene product itself but also in the expenditure which has to be incurred in terms of developing and producing the machines for the production of packaged hygiene products. Thus, the machines for the production of packaged hygiene products with the conventional end closures require either a station for applying the adhesive tape or a station for applying the separate glue, in each case linked to associated machine logistics for supplying and storing the adhesive tape or the additional glue.

III. STATEMENT OF THE INVENTION

a) Technical Object

Therefore the object of the present invention is to provide a packaged hygiene product for receiving body fluids and/or excretions and a process for the production of such a product, which on the one hand avoids the fish mouth effect and which on the other hand both makes the packaged hygiene product itself cheaper to produce and also simplifies the machines for the production of the packaged hygiene products.

b) Attainment of the Object

That object is attained by a packaged hygiene product and a process for the production of a packaged hygiene product having the features of claim 1 and claim 14 respectively. Further configurations of the present invention are set forth in the appendant claims.

In accordance with the invention there is proposed a packaged hygiene product for receiving body fluids and/or excretions, preferably from the human body, which has a body side facing towards the human body and a washing side facing away from the human body and can be folded or rolled up for packaging, wherein an adhesive is disposed at least partially at the washing side, and which includes a packaging material which has an inside facing towards the hygiene product and an outside facing away from the hygiene product, wherein the washing side of the hygiene product is fixed by means of the adhesive to the inside of the packaging material. In accordance with the invention that packaged hygiene product is characterised in that the packaging material has at least one opening through which a surface region of the inside of the packaging material can be releasably fixed by means of a portion of- the adhesive to the washing side of the hygiene product in order to hold the hygiene product in the packaged condition.

In the case of the packaged hygiene product according to the invention the inside of the packaging material which is preferably a thin flexible packaging film is fixed releasably by means of the adhesive to the washing side of the hygiene product. For the hygiene product to be used in the ordinary appropriate manner the packaging material can thus simply be pulled off by hand from the washing side of the hygiene product. Fixing of the hygiene product to the packaging material is effected by means of a surface application of glue, preferably on the washing side of the hygiene product. That surface application of glue can be arranged in a single continuous surface region of the washing side or may comprise a plurality of glue application surface portions which are not connected together.

The operation of placing the hygiene product together to form the packaged hygiene product is effected in accordance with the invention preferably by folding it up. The invention can be used in particular in relation to what are referred to as ‘quick-wrap’ products which are for example sanitary towels or panty liners which are folded into three. Folded into three in this sense means that there are altogether two fold lines, that is to say a total of two folds for packaging the hygiene product are produced and the two fold lines subdivide the hygiene product into a total of three regions. As an alternative to folding it up however the present invention can also be used if the hygiene product is rolled up for packaging purposes or is reduced in size in terms of its developed base area in some other fashion.

In the packaged hygiene product according to the invention, at the inside of the packaging material there is a region which is covered over by the washing side of the hygiene product. That region is referred to hereinafter as the cover region. As the adhesive is applied at a maximum over the entire washing side of the hygiene product, the corresponding adhesive surface region can at a maximum attain the size of the area of the cover region. The at least one opening according to the invention is arranged in the packaging material in such a way that it comes to lie in the cover region of the inside. In that respect the size and the geometry of the opening are to be so selected that that region of the washing side, which is accessible through the opening, is wetted with adhesive at least partially and preferably completely. That ensures that, after the hygiene product which is connected to the packaging material has been folded together or folded up, a delimited surface region of the inside of the packaging material can be releasably glued to the region of the washing side, which is wetted with the adhesive and which is accessible through the opening, in order to hold the packaging material closed or to hold the hygiene product overall in the packaged condition. In that respect that surface region of the inside, which is glued to the washing side of the hygiene product, is disposed outside the cover region.

As the conventional production of packaged hygiene products of the kind involved here in any case involves the application of an adhesive to the washing side of the hygiene product in order to fix the packaging material there, the present invention therefore advantageously uses the adhesive which is to be applied in any case, in order to hold the packaging of the hygiene product in the closed condition. Adhesive measures such as for example an adhesive tape or additional glueing which is to be provided separately are therefore not required in accordance with the invention.

The opening in the packaging material can involve a large number of geometries, depending on the respective situation of use. For example it is possible to envisage using elongate shapes. If the hygiene product itself, when not folded up, is also of an elongate shape, the longitudinal axis of the elongate opening can extend substantially transversely or parallel to the longitudinal axis of the hygiene product. It is also possible for the opening according to the invention to extend over the entire or almost the entire width of the hygiene product.

In addition it is also possible to provide a plurality of openings, for example in the form of two or more strip openings which extend. substantially parallel to each other. It is also possible to provide openings in the manner of a perforation which is arranged to extend in a straight line or along a curved trajectory. In particular rectangular shapes, square shapes, circular shapes, elliptical shapes or similar geometries can be envisaged in regard to the geometries of the opening or openings. If required and when using transparent packaging material the openings can also be arranged or formed by perforation in the form of instructions for use which the user can read through the packaging material, such as for example in the form of the instruction ‘PULL HERE’.

The adhesive preferably involves adhesive glue which is used by contact or spray application. In comparison with contact application, spray application affords an advantage in relation to those hygiene products which are not of a constant thickness such as for example sanitary towels. In the case of spray application, the glue, by virtue of its relatively low viscosity, is distributed uniformly over the glue-application surface, in comparatively short periods of time. That is not always the case when contact application is applied. It would also be possible for parts of the inside of the packaging material and/or the washing side of the hygiene product itself to serve as the adhesive. For example ultrasonic welding of the inside of the packaging material to the washing side of the hygiene product can be envisaged. In that respect it is merely necessary to ensure that the packaging material can be pulled off-the hygiene product by hand and without damaging the hygiene product.

In the process according to the invention for the production of a packaged hygiene product, firstly the hygiene product itself and the packaging material which is preferably cut to size are prepared. Then the washing side of the hygiene product and the inside of the packaging material are connected together releasably by means of the adhesive. Preferably prior to the washing side of the hygiene product being fixed to the inside of the packaging material, the opening is formed in the packaging material, preferably by being cut out therein. The operation of folding together, folding up or rolling up the hygiene product together with the packaging material fixed thereto is finally concluded with the step of a surface region of the inside of the packaging material being fixed to the washing side of the hygiene product through the opening.

c) Embodiment

An embodiment of the present invention is described by way of example hereinafter by means of a panty liner which is folded into three, with an end closure according to the invention without additional glueing or an additional adhesive tape. In the drawing:

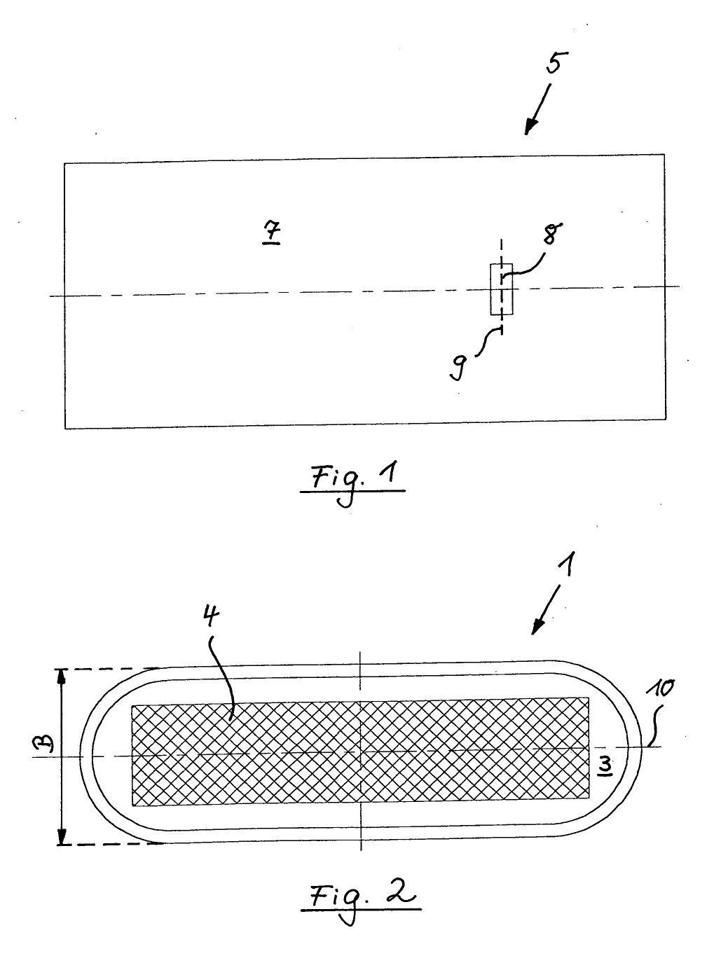

FIG. 1 shows a view on to the outside of the packaging material,

FIG. 2 shows a plan view on to the washing side of the panty liner,

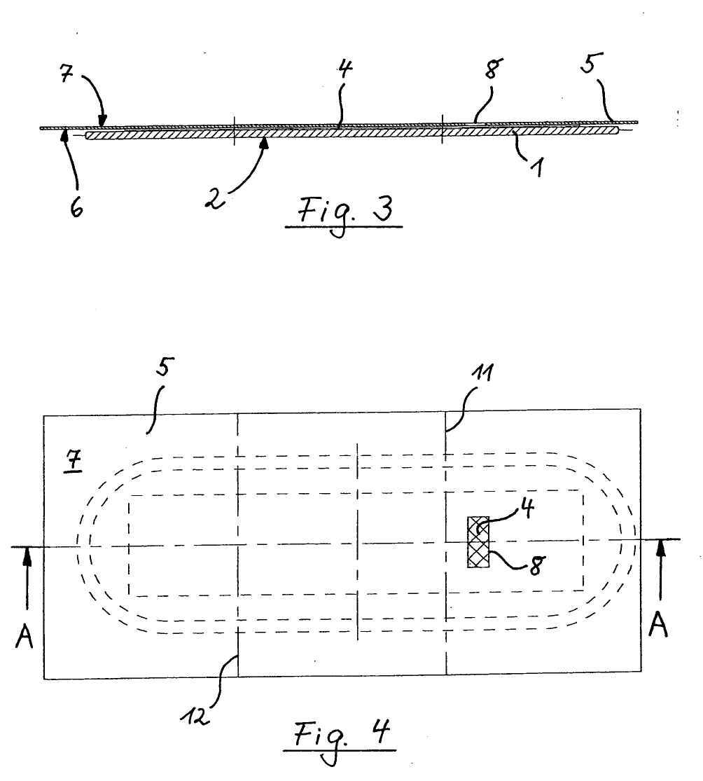

FIG. 3 shows a sectional view taken along section line A-A in FIG. 1,

FIG. 4 shows a view on to the panty liner connected to the packaging material, in the condition of not being folded up,

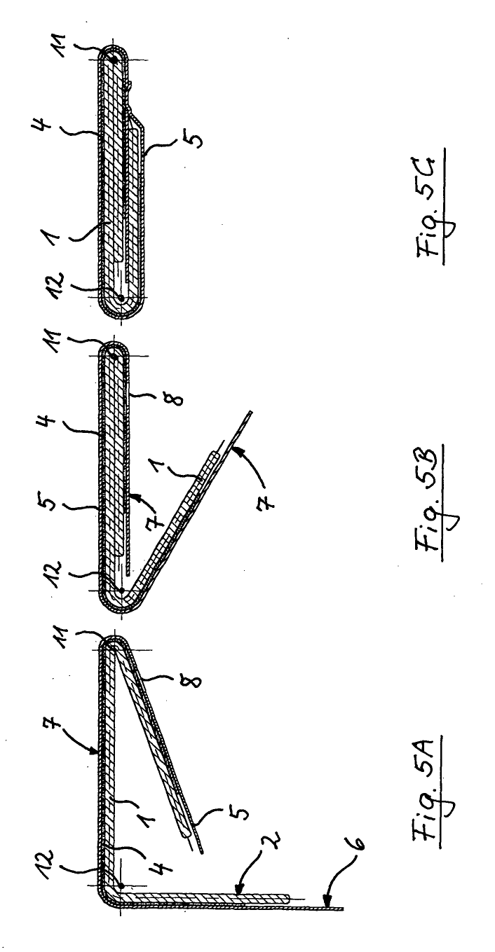

FIGS. 5A-5C show various instantaneous conditions which show the panty liner connected to the packaging material, during the folding-up procedure,

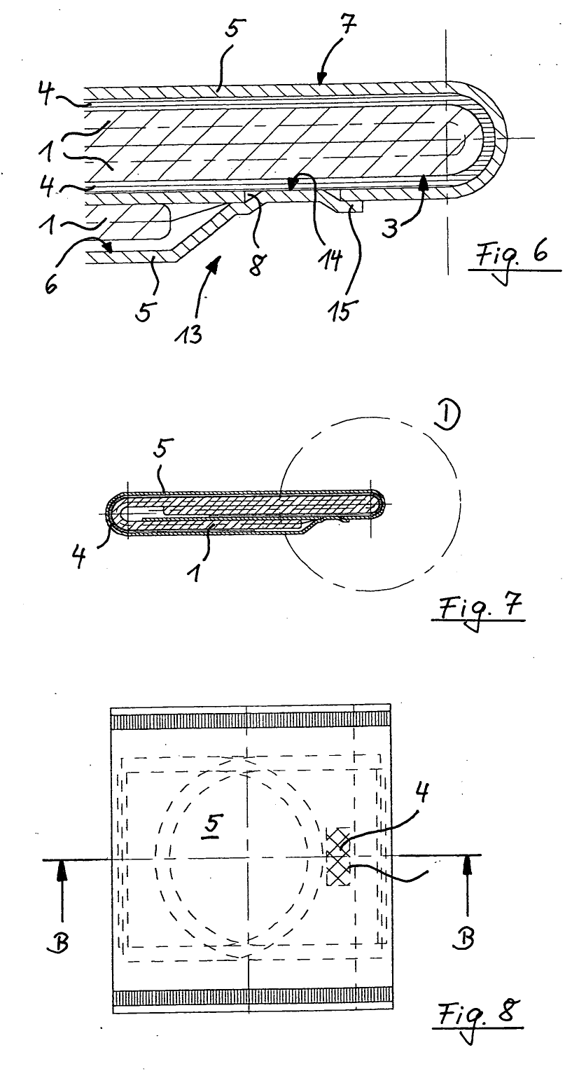

FIG. 6 shows the enlarged detail D indicated in FIG. 7,

FIG. 7 shows a sectional view taken along section line B-B in FIG. 8, and

FIG. 8 shows a view of the finished package panty liner shown in FIG. 5C from above.

FIG. 1 shows a view of the outside 7 of the packaging material 5 which is cut to size in a rectangular shape and which preferably involves a flexible or elastic packaging film of PE (polyethylene) or PP (polypropylene) of small thickness. The usual thicknesses of the flexible film are in the region of some 10 μm. As can be seen the packaging film 5 has an elongate rectangular opening 8 with a longitudinal axis 9. FIG. 2 shows a view of the washing side of a per se known panty liner 1 with rounded-off ends. A rectangular surface region of the washing side 3, which is identified by crossed hatching, is wetted continuously at its surface with an adhesive 4. If required it is also possible to wet a plurality of surface regions of the washing side 3, which are not connected together. The panty liner 1 is of a width B perpendicularly to its longitudinal axis 10.

FIGS. 3 and 4 show the panty liner 1 connected to the packaging film, wherein the panty liner 1 is shown in the view in FIG. 4 in broken lines as it is disposed underneath the packaging film 5. Also shown in broken lines in FIG. 4 is the rectangular application surface of the adhesive 4. A part of the adhesive 4 is accessible in the direction of viewing in FIG. 4, through the rectangular opening 8. The adhesive layer 4 which connects a part of the inside 6 of the packaging film 5 to a part of the washing side 3 of the panty liner 1 is shown in particular in the sectional view in FIG. 3. The body side 2 of the panty liner 1, which comes into contact with the body of the female wearer, is likewise identified in FIG. 3. The inside 6 of the packaging film 5 faces towards the panty liner 1 while the outside 7 of the packaging film 5 faces away from it. FIG. 4 makes it clear that the opening 8 is within the cover region in which the panty liner 1 covers the inside 6 of the packaging film 5.

FIGS. 5A through 5C show the operation of folding up the unit, shown in FIGS. 3 and 4, consisting of the packaging film 5, the adhesive layer 4 and the panty liner 1. As shown in FIG. 5A a first folding operation takes place about the first fold line 11. In that step two adjacent regions of the body side 2 of the panty liner 1 are caused to bear against each other, as can be seen in the end condition in that respect, as shown in. FIG. 5B. The opening 8 in the packaging film 5 is at a certain distance from the first fold line 11 and, together with the associated region of the packaging film 5, is folded through 180° out of the plane of the drawing in FIG. 4 and into same again. A second folding operation is then effected, also through 180°, about the second fold line 12. In that case the region of the panty liner 1, which is at bottom left in FIG. 5A, is laid with its body side 2 against the outside 7 of that part of the packaging film 5, which was folded over around the first fold line 11 in the course of the first folding operation. The end condition of the second folding step is shown in FIG. 5C.

FIGS. 6, 7 and 8 show the panty liner 1 which is in the finished packaged condition and which is shown in broken lines in FIG. 8. FIG. 6 shows the enlarged detail D illustrated in FIG. 7. As can be clearly seen in the lower part of FIG. 6 the one end 13 of the packaging film 5 projects beyond the associated end of the panty liner 1. In that way it is possible for a surface region 14 of the inside of the outwardly projecting end 13 of the packaging film 5 to be glued through the opening 8 to the washing side 3 of the panty liner 1. As can be seen in particular in FIG. 6, that glueing operation is effected by means of that part of the adhesive layer 4 which is accessible through the opening 8. As the adhesive layer 4 releasably joins the packaging film 5 to the panty liner 1, it is also readily possible for the surface region 14 of the end 13 of the packaging film 5 to be released from the washing side 3 to remove the panty liner 1 from the packaging film 5. For that purpose it is only necessary for the end region 15 of the end 13 of the packaging film 5, which preferably lies on the outside 7 of the packaging film 5, to be gripped and pulled by hand. The edge of the end region 15 can also be seen in FIG. 8, where it is illustrated in the form of a downwardly extending broken line.

The described embodiment clearly shows that the present invention advantageously makes use of the adhesive layer 4 which is to be applied in any case to the washing side 3. The opening 8 provided in accordance with the invention easily permits the surface region 14 of the inside 6 of the packaging film 5 to be fixed to the washing side 3 of the panty liner 1, and thus permits secure reliable packaging of the panty liner 1. In addition the panty liner 1 is enclosed in hygienically sealed relationship by the packaging film 5.

1. A packaged hygiene product for receiving body fluids and/or excretions including
the hygiene product (1) which has a body side (2) facing towards the body and a washing side (3) facing away from the body and which can be folded or rolled up for packaging, wherein an adhesive (4) is disposed at least partially at the washing side (3), and
a packaging material (5) which has an inside (6) facing towards the hygiene product (1) and an outside (7) facing away from the hygiene product (1), wherein the washing side (3) of the hygiene product (1) is fixed by means of the adhesive (4) to the inside (6),
characterised in that
the packaging material (5) has at least one opening (8) through which a surface region (14) of the inside (6) can be fixed by means of a part of the adhesive (4) to the washing side (3) in order to hold the hygiene product (1) in the packaged condition.

2. A packaged hygiene product as set forth in claim 1 characterised in that the opening (8) is of an elongate shape.

3. A packaged hygiene product as set forth in claim 2 characterised in that the hygiene product (1) is of an elongate shape and the longitudinal axis (9) of the opening (8) extends substantially transversely with respect to the longitudinal axis (10) of the hygiene product (1).

4. A packaged hygiene product as set forth in claim 2 characterised in that the hygiene product (1) is of an elongate shape and the longitudinal axis (9) of the opening (8) extends substantially parallel with respect to the longitudinal axis (10) of the hygiene product (1).

5. A packaged hygiene product as set forth in one of the preceding claims characterised in that the opening (8) extends over the entire width (B) of the hygiene product (1) or the folded hygiene product (1).

6. A packaged hygiene product as set forth in one of the preceding claims characterised in that there are provided a plurality of openings (8).

7. A packaged hygiene product as set forth in claim 6 characterised in that the openings (8) are arranged in the manner of a perforation in a straight line or in a curved trajectory.

8. A packaged hygiene product as set forth in one of the preceding claims characterised in that the opening (8) is of a rectangular shape.

9. A packaged hygiene product as set forth in one of claims 1 through 7 characterised in that the opening (8) is of a square shape.

10. A packaged hygiene product as set forth in one of claims 1 through 7 characterised in that the opening (8) is of a circular shape.

11. A packaged hygiene product as set forth in one of claims 1 through 7 characterised in that the opening (8) is of an elliptical shape.

12. A packaged hygiene product as set forth in one of the preceding claims characterised in that the adhesive (4) is adhesive glue which can be applied by means of contact application.

13. A packaged hygiene product as set forth in one of claims 1 through 11 characterised in that the adhesive (4) is adhesive glue which can be applied by means of spray application.

14. A process for the production of a packaged hygiene product (1) for receiving body fluids and/or excretions including the following steps:
providing the hygiene product (1) having a body side (2) facing towards the body and a washing side (3) facing away from the body, and
providing a packaging material (5) having an inside (6) and an outside (7),
wherein the washing side (3) of the hygiene product (1) is fixed by means of an adhesive (4) to the inside (6) of the packaging material (5) and the hygiene product (1) together with the packaging material (5) is folded or rolled up,
characterised in that
at least one opening (8) is produced in the packaging material (5) and a surface region (14) of the inside (6) is fixed through the opening (8) to the washing side (3) by means of a part of the adhesive (4) in order to hold the hygiene product (1) in the packaged condition.

15. A process as set forth in claim 14 characterised in that the operation of producing the at least one opening (8) is effected before fixing the washing side (3) to the inside (6).
